(19) United States
(12) Patent Application Publication
(10) Pub. No.: US 2025/0235625 A1
(43) Pub. Date: Jul. 24, 2025

(54) Dose Logging Device With Scale Drum Scanner

(71) Applicant: Novo Nordisk A/S, Bagsvaerd (DK)

(72) Inventor: Andre Larsen, Dragoer (DK)

(21) Appl. No.: 18/855,501

(22) PCT Filed: Apr. 24, 2023
(86) PCT No.: PCT/EP2023/060556

§ 371 Date: Oct. 9, 2024

(30) Foreign Application Priority Data
Apr. 25, 2022 (EP) 22169718.8

(51) Int. Cl. A61M 5/315

(52) U.S. Cl. CPC A61M 5/31568; A61M 5/31586; A61M 2205/3327; A61M 2205/6063

(57) ABSTRACT

A drug delivery device comprising a housing with an opening surrounded by an edge, an indicator member adapted to rotate relative to the housing during dose setting and expelling corresponding to an axis of rotation. A pattern is arranged on the indicator member and comprises a plurality of indicia indicating a rotational position of the indicator member relative to the housing. The edge comprises a scanner edge portion arranged generally in parallel with the axis of rotation. A line scanner is arranged at the scanner edge portion and adapted to scan a line formed portion of the indicator member observable in the opening as the indicator member rotates relative to the line scanner from a start rotational position to an end rotational position during dose setting and/or dose expelling thereby providing a plurality of line scan images allowing a set or expelled amount of drug to be calculated.

The present invention generally relates to arrangements allowing optical information to be both captured and observed. In a specific aspect the invention relates to medical devices and assemblies for which the generation, collecting and storing of data are relevant. In specific embodiments the invention relates to devices and systems for capturing drug delivery dose data in a reliable, user-friendly and power efficient way.

### BACKGROUND OF THE INVENTION

In the disclosure of the present invention reference is mostly made to drug delivery devices comprising a threaded piston rod driven by a rotating drive member, such devices being used e.g. in the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to people incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the notebook system requires that the patient re-members to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device itself. Addressing this problem a number of solutions have been proposed which would help a user to generate, collect and distribute data indicative of the use of a given medical device.

For example, WO 2013/120776 describes an electronic supplementary device (or “add-on module”) adapted to be releasably attached to a drug delivery device of the pen type to determine and store expelled dose amounts. The device includes a camera and is configured to perform optical character recognition (OCR) on captured images from a rotating scale drum visible through a dosage window on the drug delivery device, thereby to determine a dose of medicament that has been dialled into the drug delivery device. As the device covers the dosage window the camera is also used to capture data during dose setting, this allowing the current scale drum value to be shown on an electronically controlled display during dose setting. US 2020/0129702 and WO 2017/114769 disclose further examples of add-on devices comprising an image-capturing camera. A further external add-on device for a pen device is shown in WO 2013/004843, this device comprising a camera arrangement allowing the user to observe the scale drum during dose setting. WO 2013/120778 discloses a monitoring device in which dose size determination is based on sensors adapted to detect axial and rotational movement.

Having regard to the above, it is an object of the present invention to provide devices, assemblies and methods allowing an object to be both captured and observed in a reliable and cost-effective way. It is a further object to provide devices, assemblies and methods allowing se-cure, easy and cost-effective operation of a drug delivery assembly comprising dose logging functionality, e.g. in the form of a user-mountable add-on device.

### DISCLOSURE OF THE INVENTION

In the disclosure of the present invention, embodiments and aspects will be described which will address one or more of the above objects or which will address objects apparent from the below disclosure as well as from the description of exemplary embodiments.

The add-on device disclosed in above-mentioned WO 2013/120776 comprises a camera and is configured to perform optical character recognition (OCR) on captured images from a traditional rotating scale drum visible through a dosage window on the drug delivery device. As the device covers the dosage window the camera is also used to capture data during dose setting, which is then shown an electronically controlled display during dose setting, this adding to complexity, costs and bulk of the device. Addressing this issue WO 2013/004843 and KR 2020 0025378 disclose arrangements in which the camera system, i.e. the camera per se and the lightening means, is moved “out of the way” to allow the user to view the dosage window, however, the camera systems are still traditional and the arrangements more or less bulky.

The present invention is based on the realization that instead of using a camera to take “pictures” of a moving object, i.e. the rotating scale drum, the rotational movement may be made a functional part of the system allowing the “camera” to be reduced to a minimum in both size and complexity. As appears, in order to capture “pictures” a traditional camera sensor comprises an array of sensor elements, the array comprising a plurality of lines, each line comprising a plurality of sensor elements. Additionally, to focus a given picture on the camera surface an optical lens arrangement will in most cases have to be provided. In contrast, a line scanner is more compact and does not require an optical lens arrangement to focus the picture onto the line scanned.

Thus, in a first aspect of the invention a drug delivery assembly is provided comprising a drug reservoir or means for receiving a drug reservoir, a housing with an opening surrounded by an edge, and drug expelling means. The drug expelling means comprises a rotatable dose setting member allowing a user to set a dose amount of drug to be expelled, an indicator member adapted to rotate relative to the housing during dose setting and dose expelling corresponding to an axis of rotation, the indicator member having an initial rotational position corresponding to no dose amount being set, the amount of rotation from the initial rotational position corresponding to a set dose respectively the amount of drug remaining to be expelled from a reservoir by the expelling means, a pattern arranged circumferentially or helically on the indicator member and comprising a plurality of indicia indicating a rotational position of the indicator member relative to the housing, the indicia corresponding to a presently set dose being viewable in the opening, the edge comprising a scanner edge portion arranged generally in parallel with the axis of rotation, and a release member actuatable between a proximal position and a distal position, the proximal position allowing a dose amount to be set, the distal position allowing the drug expelling means to expel a set dose. The drug delivery assembly further comprises a line scanner coupled to the housing and arranged at the scanner edge portion and adapted to scan a line formed portion of the indicator member observable in the opening as the indicator member rotates relative to the line scanner from a start rotational position to an end rotational position during dose setting and/or dose expelling thereby providing a plurality of line scan images, as well as processor means adapted to, based on one or more line scan images captured at the beginning respectively at the end of a rotational movement of the indicator member, determine a start rotational position and an end rotational position of the indicator member and calculate a set or expelled amount of drug.

By this arrangement a simple and compact image capture device is provided which effectively allows a scale drum to be both viewed by a person and captured by electronic capturing means to determine the size of a set and/or expelled amount of a drug.

As stated above, rotational positions are determined based on one or more line scan images. When determination of a position is based on e.g. a printed scale drum numeral a composite image is created by the processor means by combining a plurality of line scan images which can then by analysed. However, scale drum information could also be provided in the form of a traditional bar code provided across the scale drum which would result in a plurality of identical line scan images being created which would not have to be combined to provide a picture.

In exemplary embodiments the scanner edge portion is arranged such that the indicia move towards or away from the scanner edge portion when a dose is being set, i.e. at “the top” or at “the bottom” of the display as viewed.

The indicator member may be provided with a code image arranged in the vicinity of the line scanner when the indicator member is in its initial rotational position, the code image thereby being scanned by the line scanner just after the indicator member is rotated away from the initial rotational position, or just before the indicator member returns to the initial rotational position. The drug delivery assembly may be prefilled with a given drug with the code image comprising information in respect of the contained drug. In the present context prefilled indicates that the drug is provided in a reservoir, e.g. a cartridge, which is not intended to be removed or refilled by the user.

Each scanned line may comprise a number of pixels, the line scanner being adapted to consecutively illuminate each pixel or a group of pixels in the line, capture pixel data with a sensor element during illumination of an individual pixel or a group of pixels and combine the pixel data to an aggregate line scan image. The line scanner may comprise a plurality of Vertical Cavity Emitting Lasers.

The above-described drug delivery assembly may comprise an add-on device adapted to be releasably mounted on a drug delivery device, the drug delivery device comprising the housing with an opening surrounded by an edge, the drug reservoir or means for receiving a drug reservoir, as well as the drug expelling means. The add-on device comprises an add-on housing adapted to be releasably attached to the drug delivery device housing in a non-moveable position, the line scanner and the processor means.

In an exemplary embodiment the add-on device further comprises an add-on dose setting member rotatable coupled to the add-on housing and adapted to engage the drug delivery dose setting member to set a dose, and an actuatable add-on release member axially moveable relative to the add-on housing and adapted to engage and axially move the drug delivery release member. The add-on dose setting member and the add-on release member may be in the form of a combined member performing both rotational and axial movement. Alternatively the add-on device may be configured to allow the drug delivery device dose setting and dose release member to be accessed directly by the user.

In a further aspect of the invention an add-on dose logging device adapted to be releasably mounted on a drug delivery device in a predefined position is provided. The drug delivery device comprises a drug reservoir or means for receiving a drug reservoir, a housing with a window surrounded by an edge, and drug expelling means comprising a rotatable dose setting member allowing a user to set a dose amount of drug to be expelled, an indicator member adapted to rotate relative to the housing during dose setting and dose expelling corresponding to an axis of rotation, the indicator member having an initial rotational position corresponding to no dose amount being set, the amount of rotation from the initial rotational position corresponding to a set dose respectively the amount of drug remaining to be expelled from a reservoir by the expelling means, a pattern arranged circumferentially or helically on the indicator member and comprising a plurality of indicia indicating a rotational position of the indicator member relative to the housing, the indicia corresponding to a presently set dose being viewable in the opening, the edge comprising a scanner edge portion arranged generally in parallel with the axis of rotation, and a release member actuatable between a proximal position and a distal position, the proximal position allowing a dose amount to be set, the distal position allowing the drug expelling means to expel a set dose. The add-on dose logging device comprises a generally tubular add-on housing defining an axis and comprising a bore with a distal opening adapted to receive the proximal end of the drug delivery device, the add-on housing comprising a distally extending lip portion with a laterally facing lip edge adapted to be arranged corresponding to a portion of the window edge when mounted, a line scanner coupled to the add-on housing and arranged at the lip edge and adapted to scan, when mounted, a line 35 formed portion of the indicator member observable in the window as the indicator member rotates relative to the line scanner from a start rotational position to an end rotational position during dose setting and/or dose expelling thereby providing a plurality of line scan images, and processor means adapted to, based on one or more line scan images captured at the beginning respectively at the end of a rotational movement of the indicator member, determine a start rotational position and an end rotational position of the indicator member and calculate a set or expelled amount of drug.

In an exemplary embodiment the add-on dose logging device further comprises an add-on dose setting member rotatable coupled to the add-on housing and adapted to engage the drug delivery dose setting member to set a dose, and an actuatable add-on release member axially moveable relative to the add-on housing and adapted to engage and axially move the drug delivery release member. The add-on dose setting member and the add-on release member may be in the form of a combined member performing both rotational and axial movement. Alternatively the add-on device may be configured to allow the drug delivery device dose setting and dose release member to be accessed directly by the user.

Each scanned line may comprise a number of pixels, the line scanner being adapted to consecutively illuminate each pixel or a group of pixels in the line, capture pixel data with a sensor element during illumination of an individual pixel or a group of pixels and combine the pixel data to an aggregate line scan image. The line scanner may comprise a plurality of Vertical Cavity Emitting Lasers.

In a further aspect a method for scanning an image comprising a plurality of pixels to produce a scan image comprising a plurality of pixel data is provided, the method comprising the steps of consecutively illuminate each pixel in the image, during illumination of an individual pixel capture pixel data with a sensor element and combine the pixel data to an aggregate scan image.

The image may comprise a single line of pixels or a plurality of lines being scanned consecutively which subsequently are combined to an aggregate multi-line scan image.

A number of pixels may be illuminated at the same time, the number being less than the number of pixels in a line. The number of sensor elements provided may be less than the number of pixels in a line. Alternatively a sensor element may be provided for each pixel in the image. The pixels may be illuminated using a Vertical Cavity Emitting Laser.

As used herein, the term “insulin” is meant to encompass any drug-containing flowable medi-cine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described dose logging assembly could also be used to create logs for other types of drug, e.g. growth hormone.

### BRIEF DESCRIPTION OF THE DRAWINGS

In the following embodiments of the invention will be described with reference to the drawings, wherein

FIG. 1A shows a pen-formed drug delivery device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIG. 4 shows a transparent representation of an add-on device with a line scanner,

FIG. 5 shows a detail of the add-on device of FIG. 4 mounted on a drug delivery device,

FIGS. 6A and 6B shows schematic representations of line scanner light source arrangements,

FIGS. 7A and 7B shows schematic representations of line scanner sensor arrangements,

FIG. 8 shows a schematic representation of scale drum indicia,

FIG. 9 shows a schematic representation of scale drum with a code image,

FIG. 10 shows the add-on device of FIG. 4 in combination with a drug delivery device,

FIG. 11 shows the add-on device of FIG. 4 mounted on a drug delivery device, and

FIG. 12 shows an add-on device with a display mounted on a drug delivery device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

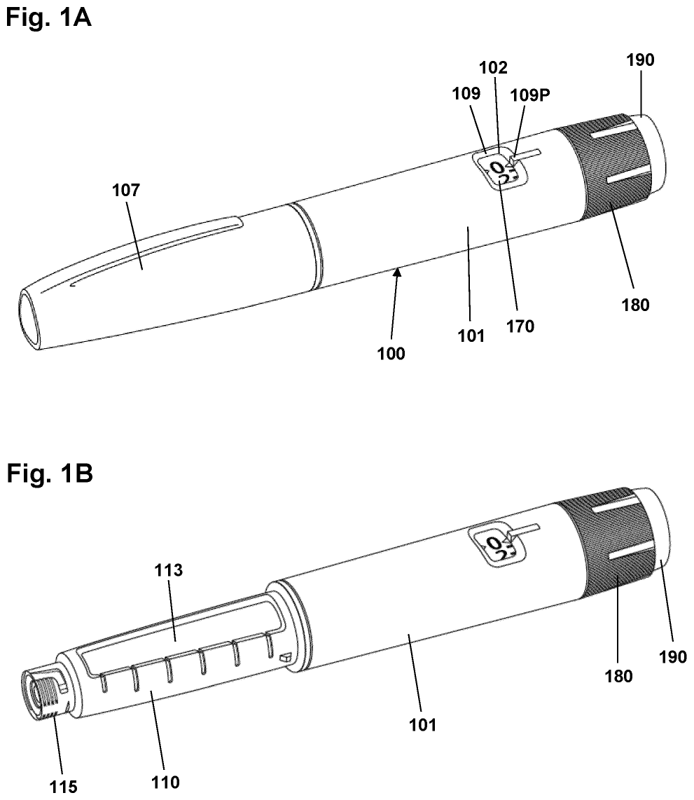

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsværd, Den-mark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 serves to manually set a desired dose of drug shown in window 102 and which can then be expelled when the button 190 is actuated. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button move proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a pre-mounted cartridge and is to be discarded when the cartridge has been emptied, in alternative embodiments the drug delivery device may be designed to allow a loaded cartridge to be re-placed, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

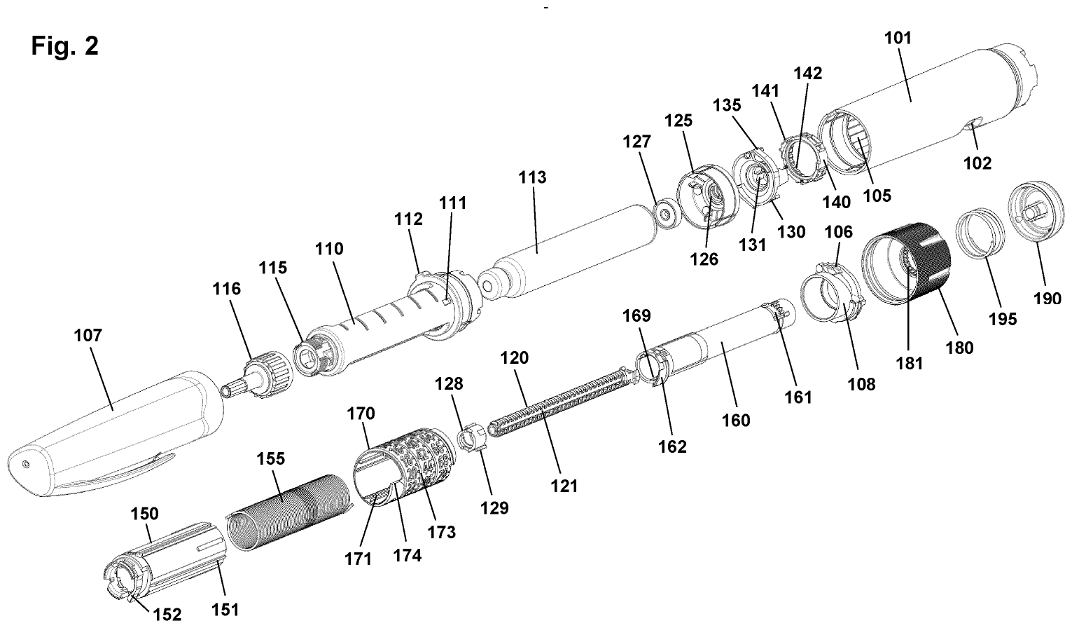

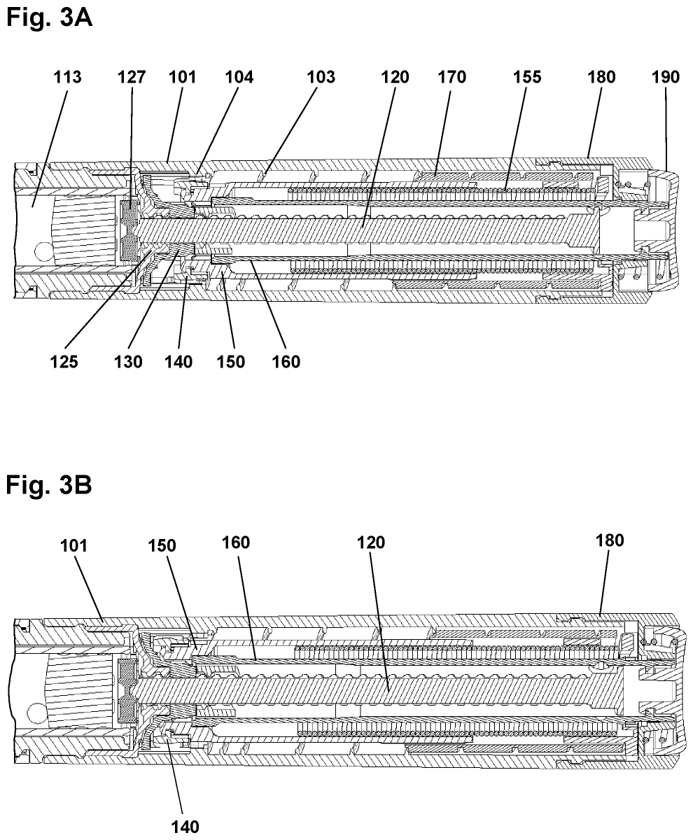

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a tabletop. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of dial ring results in a corresponding rotation of the reset tube and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system, the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled.

Turning to the dial system, the dose is set and reset by turning the dial member 180. When turning the dial, the reset tube 160, the EOC member 128, the ratchet tube 150 and the scale drum 170 all turn with it. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A dose of less than 5 IU normally cannot be paused since the rubber piston is compressed very quickly leading to a compression of the rubber piston and subsequently delivery of insulin when the piston returns to the original dimensions.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “overdose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click arm 106 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with a circumferential inner teeth structure 181 engaging a number of corresponding teeth arranged on a flexible carrier portion 161 of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

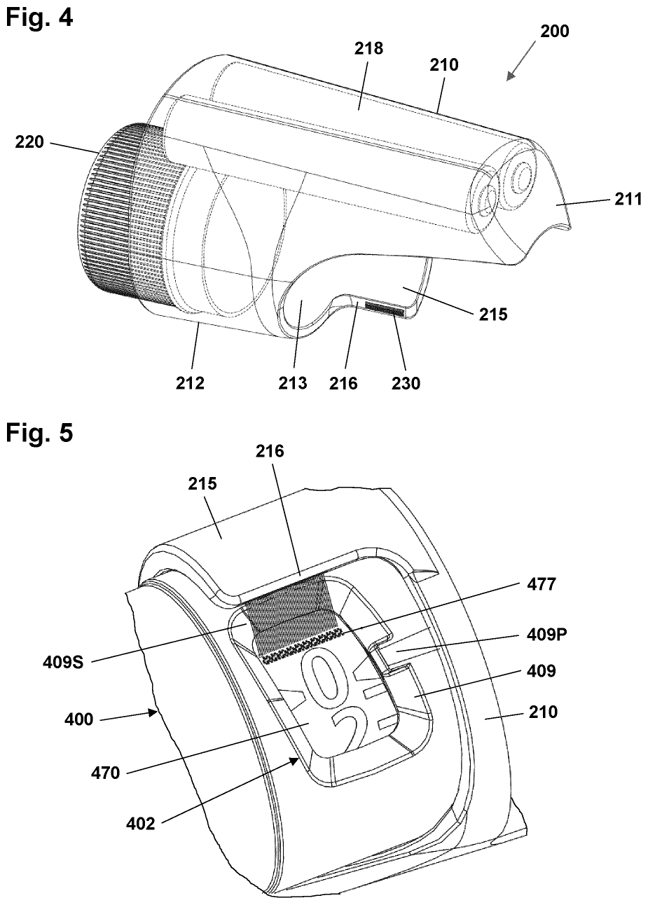

With reference to FIG. 4 an exemplary embodiment of the invention is shown in the form of an add-on dose logging device 200 adapted to be mounted on a variable dose drug delivery device of the pen type shown in FIG. 1A and thus comprising a scale drum with helically arranged dose values, the dose value being shown in the window corresponding to the actually set dose of drug to be expelled. In accordance with an aspect of the invention the add-on device comprises a line scanner having dimensions allowing it to be mounted at an edge portion of the window providing an essentially unobstructed view of the scale drum visible in the window.

More specifically, the add-on device 200 comprises a housing member 210 having a compartment portion 211 and a ring-formed portion 212 defining an axial bore having a distal opening 213 adapted to receive the proximal end of a pen device, and a combined add-on dose setting and dose release member 220 arranged in the bore proximal end and adapted to engage the pen device dose setting respectively dose release member.

The housing compartment portion is adapted to house electronic circuitry and a power source which in the shown embodiment is provided by a pair of AAAA sized replaceable batteries 218. The housing member 210 comprises a distally extending lip portion 215 with a laterally facing lip edge 216 in which a line scanner assembly 230 is arranged in communication with the electronic circuitry. The housing member further comprises releasable locking means (not shown) allowing the add-on device to be mounted axially as well as rotationally locked on the pen housing. When the housing is mounted on the pen device 400 the lip edge is positioned corresponding to the “upper” part 409S of edge 409 of the pen window 402, i.e. the edge towards which the scale drum 470 is rotated during dose setting, this allowing the user to view the scale drum during operation essentially without obstructions as shown in FIG. 5. Alternatively, when determination of rotational position is not based on the user indicia but an additional set of indicia/codes (which may not be visible to the human eye) the lip edge may be adapted to be positioned corresponding to the lower edge of the pen window 402.

The combined dose setting and dose release member 220 (in the following “combined member”) comprises a number of inwardly protruding coupling arms (not shown) adapted to non-rotationally engage corresponding coupling structures on the pen dose setting member. In the exemplary embodiment the coupling arms are flexible and adapted to engage the pen dose setting member coupling structures when the combined member is rotated initially. The combined member is biased towards a proximal-most dose setting position, e.g. by a helical spring arranged in the housing, but can be actuated in the distal direction to thereby actuate the pen dose release member. The proximally facing portion of the combined member may be provided with a display in communication with the sensor electronics, see below.

The logging device further comprises electronic circuitry adapted to determine the size of an expelled amount of drug based on determination of scale drum rotational position at the beginning respectively at the end of an out-dosing event. In accordance with an aspect of the invention a line scanner assembly (“line scanner”) 230 is arranged in the housing lip edge portion 216 and adapted to capture a line image of the scale drum corresponding to the area of the scale drum positioned just in front of the upper window edge. Based on scanned images at the beginning respectively at the end of a dose expelling event the rotational positions of the scale drum can be determined and thus the size of the expelled amount of drug. Different approaches for determining the content of the captured line images, and thus the position of the scale drum, will be discussed below.

The electronic circuitry further comprises processor means adapted to process and store the data captured by the line scanner to determine the scale drum position at the beginning respectively the end of a dosing event and thus the out-dosed amount of drug. The determined dose amount may subsequently be transmitted wirelessly to an external device, e.g. a smartphone, together with a timestamp and further data captured from a code image printed on the scale drum. Alternatively or additionally, dose data may be shown on an integrated display as shown in FIG. 12.

To save energy the electronic circuitry is generally in a sleep state when not in use and will thus have to be powered up for dose determination. Correspondingly, a “wake up” switch is provided actuated by the combined member. The switch may be actuated and the electronic circuitry including the line scanner may be turned on when the combined member is rotated by the user to set a dose of drug to be expelled. Depending on how fast the electronic circuitry powers up this would allow a code image to be read at the beginning of a dose setting procedure. Indeed, this would require the drug delivery device to be in its initial “zero state” when the user starts to set a dose. When a dose has been set the user will actuate the combined member to actuate the pen dose release member and thereby start expelling of the set dose. The axial movement of the combined member can be used to actuate an “expel” switch indicating to the electronic circuitry that out-dosing is eminent. Alternatively, if the electronic circuitry is able to power up sufficiently fast, the wake-up switch may be dispensed with and the expel switch will also serve to wake up the electronic circuitry.

If not done previously the line scanner may be turned on at this point. An end of dose state may be determined when a scale drum position corresponding to “zero” is determined. Alternatively, an end of dose state may be detected when the combined member is released and the expel switch de-activated. If the expel switch is de-activated without the end of dose state having been determined, this may indicate that the user has paused/split the dosing. Correspondingly, the electronic circuitry may be set in a paused mode waiting for a predetermined amount of time for the dose expelling to be re-started, this allowing a total amount for the split dose to be calculated. In case the expel switch is not re-actuated and the zero position is not subsequently detected, the dose event may be labeled as a partial event. Correspondingly, if a code image is neither scanned during the initial setting of a dose nor at the end of a dosing event, the dose event may be labeled as “not verified” as it is unknown what kind of drug has been expelled.

Turning to the line scanner per se, an exemplary type of a line scanner will be described.

A camera chip normally consists of an array of photodiodes and detects the amount of light reaching each of the photodiodes. By reading the values from each of the photodiodes at a given moment, a digital representation of the image is created. Some camera chips use what is commonly referred to as “rolling shutter”, which means that the camera chip reads the values from each column of photodiodes sequentially. The image values are then read in columns from left to right, resulting in an image where the left side is detected slightly before the right side, but for all practical use, the time difference is too small to cause blurriness or visible distortion of the image, even if imaged objects are moving during image capture.

In an exemplary embodiment of the present invention the above traditional mode of operation is reversed such that image capture is controlled primarily by the lightening system. More specifically, each section (pixel) of the line scan image is illuminated sequentially and the reflected light is captured by one or more photodiodes to detect the amount of reflected light from each section. By using a laser with a specific wavelength, the influence of varying natural light is significantly reduced and by applying a filter on the photodiode(s), disturbances from external light can be eliminated.

The embodiments described in the following are all based on the use of a number of Vertical Cavity Emitting Lasers (VCSEL's) which provide a very focused output beam with a narrow spectrum. An exemplary VCSEL has the dimensions 250 μm×250 μm×100 μm and emits light with a specific wavelength, e.g. a wavelength of 860 nm, just beyond the wavelength visible to the human eye. Light with this wavelength is not commonly present under normal light condition in a significant amount and thus is it safe to assume that a given area in front of the VCSEL is only illuminated by light with that wavelength if the VCSEL is “on” and emitting light. VCSEL's are produced in large quantities on wafers and mounted on chips, which can then be mounted on printed circuit boards (PCB's). VCSEL's first appeared in smartphones, integrated in the autofocus and proximity sensing applications.

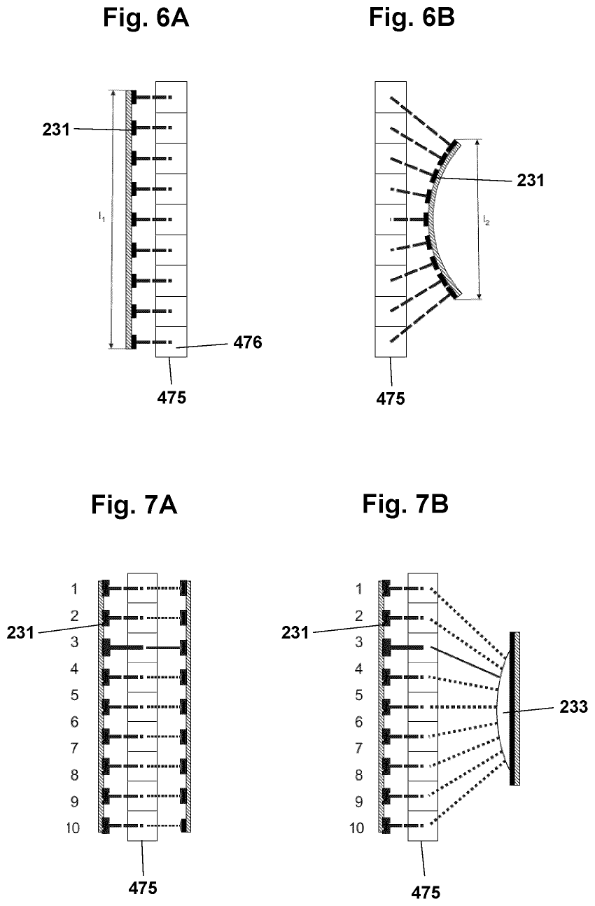

The concept of sequentially illuminating a scale drum line image 275 requires a number of VCSEL's placed such that they can each illuminate a specific section/pixel 276 of the line to be captured. As shown in FIG. 6A the VCSEL's 231 can be placed with a spacing equivalent to the required resolution of the line scanner or as shown in FIG. 6B closer to each other and angled to provide the required resolution at a given distance.

The detection if the reflected light may be done using a dedicated photodiode 232 for each section and paired with a corresponding VCSEL as shown in FIG. 7A, such that the pair of VCSEL and photodiode is activated simultaneously in sequence along the row they a placed in. As photodiodes often have a much wider opening angle than the beam width of a VCSEL, a single photodiode 233 (or a few) may be used to detect the light from several VCSEL's in the line or from all of the VCSEL's in the line of VCSEL's as shown in FIG. 7B.

In the add-on dose logging device 200 of FIG. 4 the line scanner is arranged such that the scale drum is visible to both the VCSELs and the photodiodes. As the appearance of the scale drum is known, a table based on domain knowledge can be created. Alternatively, all the scanned lines can be combined to create an image and through OCR (Optical Character Recognition) determine the numbers having passed the line scanner and determine the dose setting from which it started and where it ended and calculate the given dose.

If using domain knowledge the line scanner reads the combination of light and dark sections/pixels by simply determining a high or low state in each section/pixel in the current line and thereby creates a digital code for that line. By scanning a complete dial drum, a table of digital codes for each increment (line height) can be established. As the scale drum has blank sections between the numbers, the line scanner will not be able to distinguish different lines in those areas from each other. However, as a new number gets within view of the line scanner, the system will know how far the drum has turned since last positively identified position and then current position.

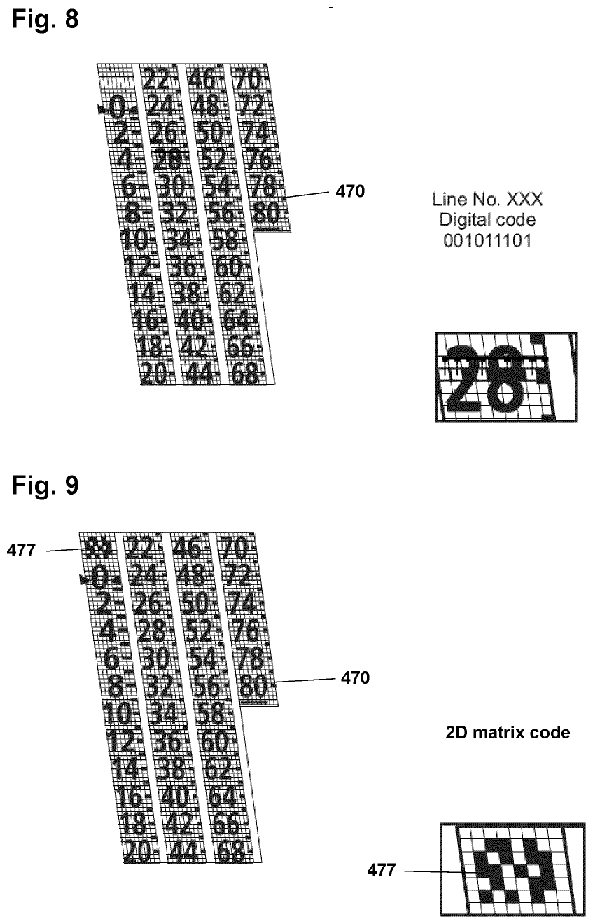

FIG. 8 shows a schematic representation of a scale drum 470 adapted to be read by a line scanner with a resolution of 9 pixels per line. In the figure a digital code for a given line through a portion of the numeral “28” is shown. In the schematic example shown the number of pixels are very low providing a digital code which most likely will not be unique, however, for an actual implementation in a dose logging device the resolution would be much higher. Additionally, the configuration of the numerals may be modified to make each read line unique. To achieve the latter additional markings may be added which may be visible or non-visible to the human eye. Alternatively, the entire line coding may be provided by invisible markings without relying on the visible indicia provided for the user.

As the appearance of the dial drum is known, the reading of digital codes for different lines can be presumed to occur in a specific order, which can be used both during error correction to improve system reliability and to save power. When the dial drum starts rotating and digital codes are read in sequence, the system may turn off a number of VCSEL's and only read the first section of the digital code and verify that that the digital codes expected to be read also appears to be read. If a bad/unexpected code-section is read due to error, the system can switch all VCSEL's on again and reconfirm actual position of the dial drum. Similarly, the system can turn all VCSEL's on if it is known that more than one area of the drum have identical first sections of their digital codes.

Domain knowledge also improve system redundancy if one or more entire lines are missed during a scanning, as the rotational distance between two parts of a data stream spaced apart can easily be reconstructed. Since the position reading is absolute, only the start and end codes are actually necessary to calculate the rotation of the dial drum and thus the size of the dose. However, by reading a steady stream of scale drum positions, not only the size but also the speed of the out-dosing can be determined. This can be used to detect (and subtract) air shots and priming events. Warnings or notifications can be issued if a partially blocked needle cause the out-dosing rate to be unusually low.

It respect of domain knowledge it should be noted that the read position does not correspond to the actually set position, i.e. the nominal dose size number positioned in the center of the window corresponding to the dose pointer 409P, but is off-set by around 2 units. Indeed, that does not pose a problem, however, as the initial portion of the scale drum, i.e. above the zero, is blank, it would be necessary to add additional information to that portion of the scale drum allowing the line scanner to read a code corresponding to a set dose size of e.g. 1 unit. That could be in the form of a code image 477 added for an additional purpose (see below) or solely for position determination. Such an additional code may be visible as shown in FIG. 9 or non-visible to the human eye.

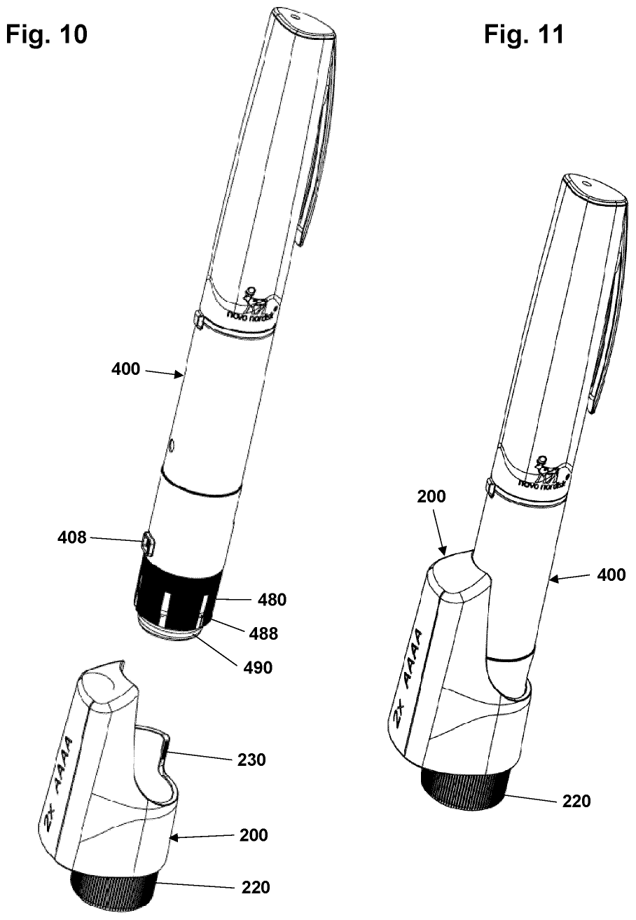

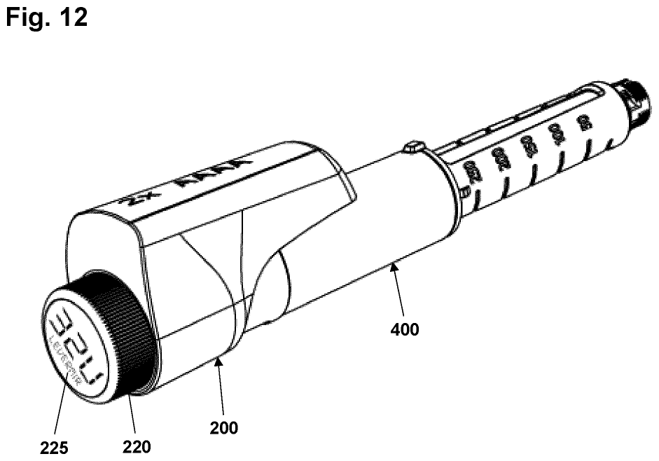

In a situation of use, see FIGS. 10 and 11, the user mounts the add-on device 200 on the proximal end of a corresponding pen drug delivery device 400, the add-on releasable locking means engaging a corresponding locking knob 408 on the pen housing 410, whereby the pen dose setting member 480 is received in the combined member 220. Depending on the design of the latter two members they may become rotationally locked during the mounting operation or, as in the present embodiment, subsequently when the user starts to set a dose and the flexible coupling arms non-rotationally engage corresponding coupling grooves 488 on the pen dose setting member.

To set a dose the user rotates the outer add-on dose setting member 220 until the desired dose size is shown in the pen display window. During initial rotation of the flexible coupling arms will slide on the pen dose setting member until the arms non-rotationally engage the corresponding axially oriented coupling grooves 488 on the pen dose setting member.

When a desired dose is set the user pushes the combined member distally moving it into engagement with the pen release button 490 which starts to move distally and subsequently releases the spring-loaded expelling mechanism. During dose expelling the electronic circuitry will determine the amount of rotation of the scale drum as described above and thereby the expelled dose.

When a set dose is expelled, or the user desires to pause injection, the user releases pressure on the combined member which is the biased towards its initial proximal-most position. When the size of an expelled dose amount has been successfully determined the dose size as well as additional data, e.g. time-stamp and drug type, may be transmitted wirelessly to the user's smartphone and/or shown on the logging device display 225 in case the latter is provided, this as shown in FIG. 12.

In the shown embodiment a 2D matrix code 477 (see FIG. 5) is printed on the initial portion of the scale drum, providing the logging device with information of e.g. device type, drug type- and concentration, batch-number and other relevant information. The code can be printed on the scale drum after assembly of the device, such that a common scale drum can be used for all devices. Information from the 2D matrix code may be relayed to a database through the user's smartphone to verify the authenticity of the prefilled device being used.

If the logging device is in direct or indirect communication with such a database, the database can keep track of use of the devices. In the event of counterfeiters refiling devices or producing counterfeit devices, use of irregular numbered devices or already used devices reappearing in use, can be detected and the user warned. Such warnings may be displayed on the reading unit display or on the user's smartphone app.

In the above description of exemplary embodiments the logging device has been described in the form of an add-on device adapted to capture dose data from a drug delivery device to which is has been mounted, however, the described line scanner solution may alternatively be incorporated in a unitary device.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery assembly comprising:
a drug reservoir or means for receiving a drug reservoir,
a housing with an opening surrounded by an edge,
drug expelling means comprising:
a rotatable dose setting member allowing a user to set a dose amount of drug to be expelled,
an indicator member adapted to rotate relative to the housing during dose setting and dose expelling corresponding to an axis of rotation, the indicator member having an initial rotational position corresponding to no dose amount being set, the amount of rotation from the initial rotational position corresponding to a set dose respectively the amount of drug remaining to be expelled from a reservoir by the expelling means,
a pattern arranged circumferentially or helically on the indicator member and comprising a plurality of indicia indicating a rotational position of the indicator member relative to the housing, the indicia corresponding to a presently set dose being viewable in the opening, the edge comprising a scanner edge portion arranged generally in parallel with the axis of rotation, and
a release member actuatable between a proximal position and a distal position, the proximal position allowing a dose amount to be set, the distal position allowing the drug expelling means to expel a set dose,
a line scanner coupled to the housing and arranged at the scanner edge portion and adapted to scan a line formed portion of the indicator member observable in the opening as the indicator member rotates relative to the line scanner from a start rotational position to an end rotational position during dose setting and/or dose expelling thereby providing a plurality of line scan images, and
processor means adapted to, based on one or more line scan images captured at the beginning respectively at the end of a rotational movement of the indicator member, determine a start rotational position and an end rotational position of the indicator member and calculate a set or expelled amount of drug.

2. The drug delivery assembly as in claim 1, wherein the scanner edge portion is arranged such that the indicia move towards or away from the scanner edge portion when a dose is being set.

3. The drug delivery assembly as in claim 1, wherein the indicator member comprises a code image arranged in the vicinity of the line scanner when the indicator member is in its initial rotational position, the code image thereby being scanned by the line scanner:
(i) just after the indicator member is rotated away from the initial rotational position, or
(ii) just before the indicator member returns to the initial rotational position.

4. The drug delivery assembly as in claim 3, wherein the drug delivery assembly is prefilled with a given drug, the code image comprises information in respect of the contained drug.

5. The drug delivery assembly as in claim 1, wherein each scanned line comprises a number of pixels, the line scanner being adapted to:
consecutively illuminate each pixel or a group of pixels in the line,
during illumination of an individual pixel or a group of pixels capture pixel data with a sensor element, and
combine the pixel data to an aggregate line scan image.

6. The drug delivery assembly as in claim 1, wherein:
the line scanner comprises a plurality of Vertical Cavity Emitting Lasers.

7. The drug delivery assembly as in claim 1, wherein the drug delivery assembly is in the form of a unitary device.

8. The drug delivery assembly as in claim 1, comprising an add-on device adapted to be releasably mounted on a drug delivery device, the drug delivery device comprising:
the housing with an opening surrounded by an edge,
the drug reservoir or means for receiving a drug reservoir, and
the drug expelling means,
the add-on device comprising:
an add-on housing adapted to be releasably attached to the drug delivery device housing in a non-moveable position,
the line scanner, and
the processor means.

9. The drug delivery assembly as in claim 8, the add-on device further comprising:
an add-on dose setting member rotatable coupled to the add-on housing and adapted to engage the drug delivery dose setting member to set a dose, and
an actuatable add-on release member axially moveable relative to the add-on housing and adapted to engage and axially move the drug delivery release member.

10. The drug delivery assembly as in claim 9, wherein the add-on dose setting member and the add-on release member is in the form of a combined member.

11. An add-on dose logging device adapted to be releasably mounted on a drug delivery device in a predefined position, the drug delivery device comprising:
a drug reservoir or means for receiving a drug reservoir,
a housing with a window surrounded by an edge,
drug expelling means comprising:
a rotatable dose setting member allowing a user to set a dose amount of drug to be expelled,
an indicator member adapted to rotate relative to the housing during dose setting and dose expelling corresponding to an axis of rotation, the indicator member having an initial rotational position corresponding to no dose amount being set, the amount of rotation from the initial rotational position corresponding to a set dose respectively the amount of drug remaining to be expelled from a reservoir by the expelling means,
a pattern arranged circumferentially or helically on the indicator member and comprising a plurality of indicia indicating a rotational position of the indicator member relative to the housing, the indicia corresponding to a presently set dose being viewable in the opening, the edge comprising a scanner edge portion arranged generally in parallel with the axis of rotation, and
a release member actuatable between a proximal position and a distal position, the proximal position allowing a dose amount to be set, the distal position allowing the drug expelling means to expel a set dose,
the add-on dose logging device comprising:
a generally tubular add-on housing defining an axis and comprising a bore with a distal opening adapted to receive the proximal end of the drug delivery device, the add-on housing comprising a distally extending lip portion with a laterally facing lip edge adapted to be arranged corresponding to a portion of the window edge when mounted,
a line scanner coupled to the add-on housing and arranged at the lip edge and adapted to scan, when mounted, a line formed portion of the indicator member observable in the window as the indicator member rotates relative to the line scanner from a start rotational position to an end rotational position during dose setting and/or dose expelling thereby providing a plurality of line scan images, and
processor means adapted to, based on one or more line scan images captured at the beginning respectively at the end of a rotational movement of the indicator member, determine a start rotational position and an end rotational position of the indicator member and calculate a set or expelled amount of drug.

12. The add-on dose logging device as in claim 11, further comprising:
an add-on dose setting member rotatable coupled to the add-on housing and adapted to engage the drug delivery dose setting member to set a dose, and
an actuatable add-on release member axially moveable relative to the add-on housing and adapted to engage and axially move the drug delivery release member.

13. The add-on dose logging device as in claim 12, wherein the add-on dose setting member and the add-on release member is in the form of a combined member.

14. The add-on dose logging device as in claim 1, wherein each scanned line comprises a number of pixels, the line scanner being adapted to:
consecutively illuminate each pixel or a group of pixels in the line,
during illumination of an individual pixel or a group of pixels capture pixel data with a sensor element, and
combine the pixel data to an aggregate line scan image.

15. The add-on dose logging device as in claim 11, wherein:
the line scanner comprises a plurality of Vertical Cavity Emitting Lasers.
